(19) United States
(12) Patent Application Publication
(10) Pub. No.: US 2024/0016756 A1
(43) Pub. Date: Jan. 18, 2024

(54) Therapeutic Foot Patch For Cough Treatment

(71) Applicant: ASO LLC, Sarasota, FL (US)

(72) Inventors: Brooke A. Hagerty, Bradenton, FL (US); Edmund A. Sinda, Bradenton, FL (US)

(21) Appl. No.: 18/221,943

(22) Filed: Jul. 14, 2023

Related U.S. Application Data
(60) Provisional application No. 63/389,471, filed on Jul. 15, 2022.

(51) Int. Cl. A61K 9/70

(52) U.S. Cl. CPC A61K 9/7038

(57) ABSTRACT

A method of treating cough includes applying a therapeutic foot patch to a bottom of a foot. The therapeutic foot patch has a flexible backing with a skin contact side that contacts the bottom of the foot. An active ingredient carrier section on the skin contact side carries a cough-reducing active ingredient that imparts a chemesthetic sensation to the foot.

### CROSS-REFERENCE TO RELATED APPLICATION

This claims the benefit of priority to Application No. 63/389,471, filed Jul. 15, 2022, which is incorporated by reference in its entirety.

### FIELD

This relates to the field of cough treatment and, more particularly, treating cough using topical patches.

### BACKGROUND

Cough and nasal congestion are common reasons for visiting a physician, which is one reason over-the-counter (“OTC”) decongestant and cough suppressant products are widely available. The majority of these products are taken orally as drugs that target cold and flu symptoms. Unfortunately, young children and people with certain medical conditions may not be able to take drugs that target cold and flu symptoms because they contain a cough suppressant, an analgesic, and a decongestant.

An alternative approach to treating cough and congestion is to apply a topical ointment containing aromatic active ingredients such as menthol, camphor, and eucalyptus to the chest and neck. These ointments deliver a cooling sensation to the nasal passages, but they are often greasy and sticky. Many people will not use these ointments because they consider them messy or consider the aromatic vapors to be undesirable.

### BRIEF SUMMARY

It would be beneficial to have a topical composition that uses cough-reducing active ingredients but does not have to be administered to the chest or neck. The therapeutic foot patch described here carries a cough-reducing active ingredient, but can be applied to a human foot.

An example of a method includes treating cough in a human by applying a therapeutic foot patch to a bottom of the human's foot. The therapeutic foot patch includes a flexible backing having an adhesive skin contact side that is adhered to the bottom of the foot and an active ingredient carrier section on the adhesive skin contact side. The active ingredient carrier section carries a cough-reducing active ingredient that imparts a chemesthetic sensation to the foot.

This method may also include one or more of the following features.

In certain examples, the active ingredient carrier section does not contact a heel of the foot or balls of the foot.

In certain examples, the flexible backing does not contact a heel of the foot or balls of the foot.

The human may be child.

The cough-reducing active ingredient may be within a liquid absorbent material on the active ingredient carrier section.

The therapeutic foot patch may be applied to a bottom of the human's big toe.

The adhesive skin contact side may include adhesive along an outer perimeter of the therapeutic foot patch and the active ingredient carrier section may be surrounded by the adhesive.

The method may further include, immediately prior to applying the therapeutic foot patch to the bottom of the human's foot, placing the cough-reducing active ingredient on the active ingredient carrier section.

The method may further comprise strapping the therapeutic foot patch to the human foot by wrapping a strap of the therapeutic foot patch around a midfoot region of the foot.

The active ingredient carrier section may be on a medial longitudinal arch of the foot when applied to the foot.

Another example of a method includes treating cough in a human by applying a therapeutic foot patch to a bottom of the human's foot. The therapeutic foot patch includes a flexible backing having a non-adhesive skin contact side that contacts the bottom of the foot. An active ingredient carrier section is on the non-adhesive skin contact side. The active ingredient carrier section carries a cough-reducing active ingredient that imparts a chemesthetic sensation to the foot. At least one strap wraps around the foot and attaches the therapeutic foot patch thereto.

This method may also include one or more of the following features.

In certain examples, the active ingredient carrier section does not contact a heel of the foot or balls of the foot.

In certain examples, the flexible backing does not contact a heel of the foot or balls of the foot.

The human may be a child.

The cough-reducing active ingredient may be within a liquid absorbent material on the active ingredient carrier section.

The method may also include, immediately prior to applying the therapeutic foot patch to the bottom of the human's foot, placing the cough-reducing active ingredient on the active ingredient carrier section.

The method may also include strapping the therapeutic foot patch to the human foot by wrapping a strap of the therapeutic foot patch around a midfoot region of the foot.

The active ingredient carrier section may be on a medial longitudinal arch of the foot when applied to the foot.

The at least one strap may include a pair of straps that wrap around the foot and attach the therapeutic foot patch thereto.

The therapeutic foot patch may form the shape of an animal with arms and legs where the arms and legs form the at least one strap.

### BRIEF DESCRIPTION OF THE DRAWINGS

FIG. 1 is a first example of the foot patch attached to the bottom of a human foot.

FIG. 2 is a skin contact side view of the first example of the foot patch of FIG. 1.

FIG. 3 is an outer surface view of the foot patch of FIG. 1.

FIG. 4 is a side view of the foot patch of FIG. 1.

FIG. 5 is a diagram of a second example of the foot patch attached to the bottom of a human foot.

FIG. 6 is a side view of the foot patch of FIG. 5 attached to the bottom of the human foot.

FIG. 7 is a third example of the foot patch attached to a human toe.

FIG. 8 is a perspective view of the foot patch of FIG. 7 attached to the human toe.

FIG. 9 is a fourth example of the foot patch attached to the bottom of a human foot.

FIG. 10 is an outer surface view of other possible examples of the foot patch.

FIG. 11 is an outer surface view of an example of a foot patch including straps.

FIG. 12 is a bottom view of a human foot wearing the foot patch of FIG. 11.

FIG. 13 is a side perspective view the human foot wearing the foot patch of FIG. 11.

FIG. 14 is an outer surface view of two additional examples of the foot patch including straps.

FIG. 15 is a side perspective view of a human foot wearing another example of the foot patch including straps.

FIG. 16 is a side perspective view of a human foot wearing another example of the foot patch including straps.

### DETAILED DESCRIPTION

This disclosure describes examples, but not all possible examples of the therapeutic foot patch and related methods. Where a particular feature is disclosed in the context of a particular example, that feature can also be used, to the extent possible, in combination with and/or in the context of other examples. The therapeutic foot patch and related methods may be embodied in many different forms and should not be construed as limited to only the features and examples described here.

The therapeutic foot patch is used in a method of treating cough in a human by applying the foot patch to the bottom of a human foot. The foot patch carries at least one active ingredient that interacts with the nerves in the sole or bottom of the human foot to relieve symptoms of cold and/or flu such as cough. The active ingredient may include one or more compounds that impart a chemesthetic sensation to the wearer. A chemesthetic sensation occurs when a compound modulates nerve receptors associated with other sensations such as pain, touch, and temperature. In this case, the active ingredient modulates nerve receptors associated with chemesthetic sensations in the foot.

Such nerve receptors may, in some examples, involve transient receptor potential channels (“TRP”). TRPs act as cellular sensors and respond to changes in the external environment. Bonvini, et al., “Targeting TRP channels for chronic cough: from bench to bedside,” Naunyn-Schmiedeberg's Arch Pharmacol, Vol. 388, pgs. 401-420 (2015). One particular TRP, called TRPM8, is associated with the perception of a cooling sensation. It has been proposed that compounds such as menthol, icilin, and eucalyptol provide a cooling effect to skin via the TRPM8 channels. Id. Other natural compounds such linalool, geraniol are also known to activate the TRPM8 channels. Camphor has also been show to modulate TRP activity. Nguyen, et al., “Structural basis for promiscuous action of monoterpenes on TRP channels,” Communications Biology, https://doi.org/10.1038/s42003-021-01776-0 (2021). There are many other natural and synthetic compounds that may be used as the active ingredient.

The active ingredient may be part of a formulation including the active ingredient and one or more acceptable excipients. The formulation may be therapeutically effective for reducing coughing. Examples of the formulation may include the active ingredient in a suitable liquid, liquid-like, wax, or wax-like carrier. Such carriers may include water, oil, oil-based, wax, or wax-based materials in which the active ingredient is dissolved or dispersed and may have a low viscosity similar to water or a high viscosity similar to a gel or waxy substance. Certain examples of the formulation include essential oils and petrolatum-based products.

The formulation includes a cough-reducing effective amount of the active ingredient. In certain examples, the foot patch 100 carries 0.1-10 grams, 0.1-7 grams, 0.1-5 grams, or 0.1 to 2 grams of the formulation. In certain examples, the formulation includes 1%-90% w/w, 1%-75% w/w, 1%-50% w/w, 1%-30% w/w, 1%-20% w/w, or 1%-10% w/w of the active ingredient(s).

The cough-reducing effective amount of the active ingredient may be adjusted based on empirical observations by a medical professional, by size of the wearer's foot, by age of the desired user, or by the desired active ingredient strength of the foot patch.

The foot patch may be applied once per day or multiple times per day while symptoms are present. One foot patch may be reused multiple times or a new foot patch may be used each time the wearer desires treatment.

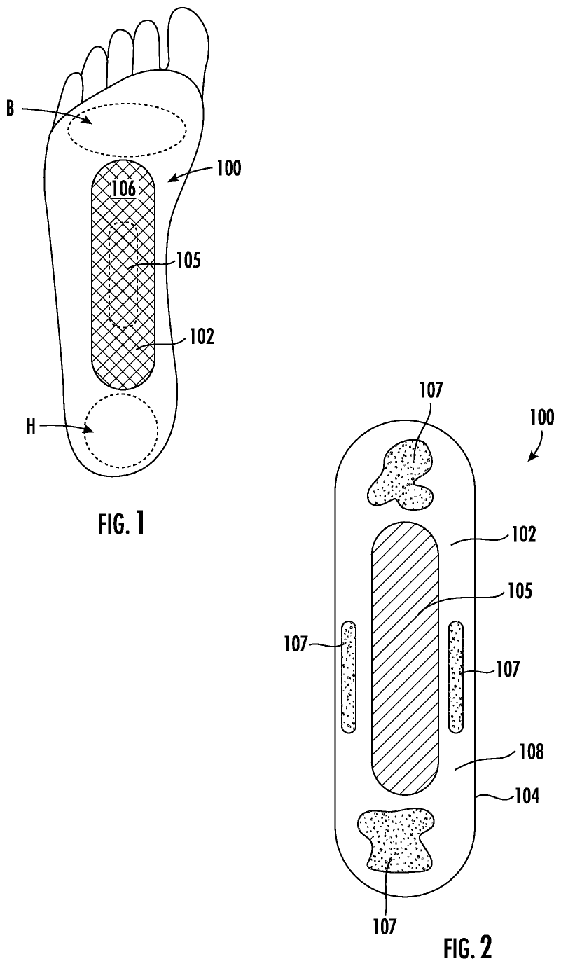

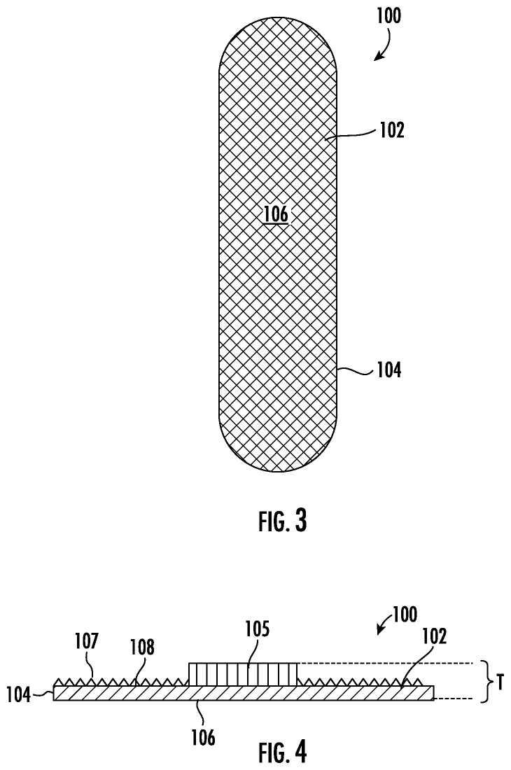

Referring to FIGS. 1-4, a first example of the therapeutic foot patch 100 includes a backing 102, an active ingredient carrier section 105, and an adhesive 107. When the active ingredient carrier section 105 is drawn with a dashed outline, the dashed outline represents the location of active ingredient carrier section 105 under the surface. In this and all subsequent examples of the foot patch 100 described herein, the same references numerals are used to refer to the corresponding component of the example being shown and described.

The backing 102 may be made of flexible woven or non-woven fabric or polymeric material. Such a material may include, for example, polyester, polyethylene, polypropylene, polyurethane, or the like. In other examples, the material may be a foam, silicone, or the like.

The backing 102 provides the physical structure of the patch and, therefore, is preferably thin and comfortable, yet durable enough to be worn the foot for an extended time.

The backing 102 includes an outer perimeter 104 defining the size and shape of the patch 100, an outer surface 106, and a skin contact surface 108 opposite the outer surface 106. In this example, the patch 100 is sized to adhere to the bottom of the foot. In other examples, the patch 100 and its components may be larger, smaller, or differently shaped for use with different sizes of feet an placement on different parts of the foot.

The adhesive 107 is designed to be placed against the sole of the foot and adhere the patch 100 to the skin. The adhesive 107 may be selected from skin contact adhesives that can adhere firmly to the skin, but are removable without damaging the skin. Examples of skin contact adhesives include, acrylic adhesives, silicone adhesives, hydrogels, hydrocolloids, silicone or the like. The adhesive material may also include an additive that provides benefits to the skin such as vitamins, vitamin E, and/or zinc oxide, for example. The adhesive 107 may cover substantially all or just a portion of the skin contact surface 108.

The foot patch 100 is preferably flexible and able to conform to the contours of the wearer's foot so that it is comfortable and will not substantially interfere with the wearer's activities. In certain examples, the foot patch 100 has a thickness T of 0.1 mm to 100 mm, 0.1 mm to 75 mm, mm to 50 mm, or 0.1 mm to 25 mm.

The active ingredient carrier section 105 may have many different forms, depending on the desired application. In certain examples, the active ingredient carrier section 105 is a portion of the formulation that is applied manually by the wearer using an applicator to the active ingredient carrier section 105. In this case, the wearer can select the formulation the wearer desires to use and apply the desired formulation to the active ingredient carrier section 105. The formulation may be obtained from a formulation container and an amount of formulation is applied to the active ingredient carrier section 105 using an applicator such as a cotton swab, a finger, a dropper, a brush, or the like.

In another example, the formulation is distributed within a liquid absorbent material that is attached to the active ingredient carrier section 105. The liquid absorbent material is capable of absorbing the formulation and holding the formulation therein. Examples of liquid absorbent materials include polyester, lyocell, wool, cotton, nylon, hydrocolloid, hydrogel, bamboo, microfiber, lycra, polyurethane, foam, or the like. The liquid absorbent material may also be capable of maintaining the formulation when the wearer is standing or wearing socks and/or shoes.

In yet another example, the formulation is distributed within a gel that is attached to the active ingredient carrier section 105. The gel is capable of containing the formulation and holding the formulation therein. Examples of gel materials include hydrocolloids, hydrogels, or the like.

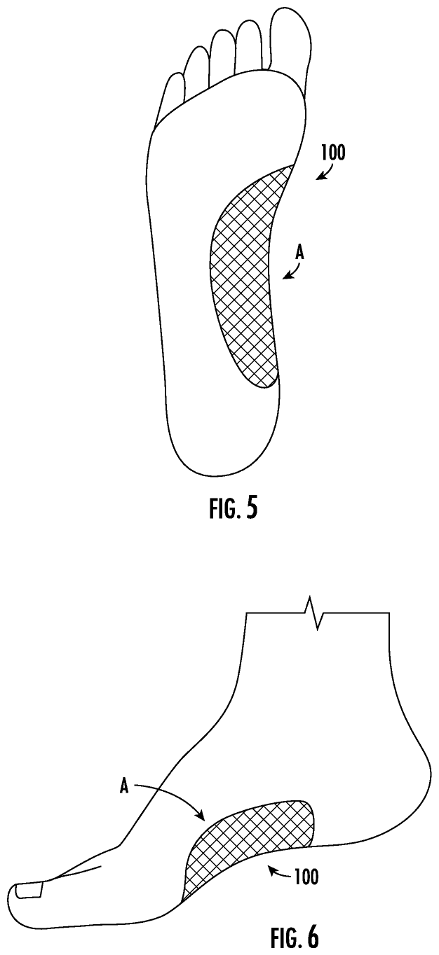

Referring to FIGS. 5 and 6, a second example of the foot patch 100 is configured to be placed on the medial longitudinal arch A of the foot. In this example, the foot patch 100 does not contact the ball of the foot or the heel because the skin on the ball of the foot and heel is often thick and calloused. In contrast, the skin on the medial longitudinal arch A is typically thin and non-calloused and may provide superior transdermal contact between the nerves of the foot and the active ingredient(s).

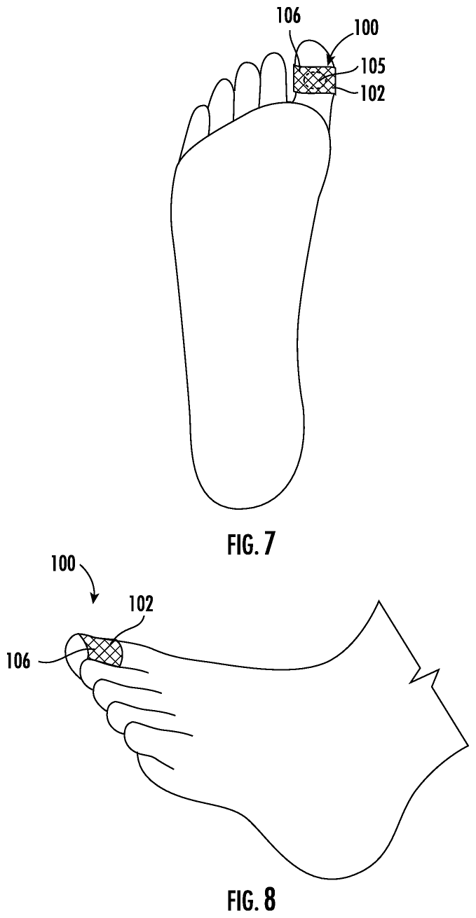

Referring to FIGS. 7 and 8, a third example of the foot patch 100 is designed to be applied to a toe of the wearer. In this example, the foot patch 100 is adhered laterally across the wearer's big toe in such a way that the active ingredient carrier section 105 is adhered against the skin on the bottom of the toe. To assist with maintaining the position of the foot patch 100 on the toe, the backing 102 wraps around the toe such that the adhesive 107 adheres to the outer surface 106 where the opposed ends of the backing 102 overlap.

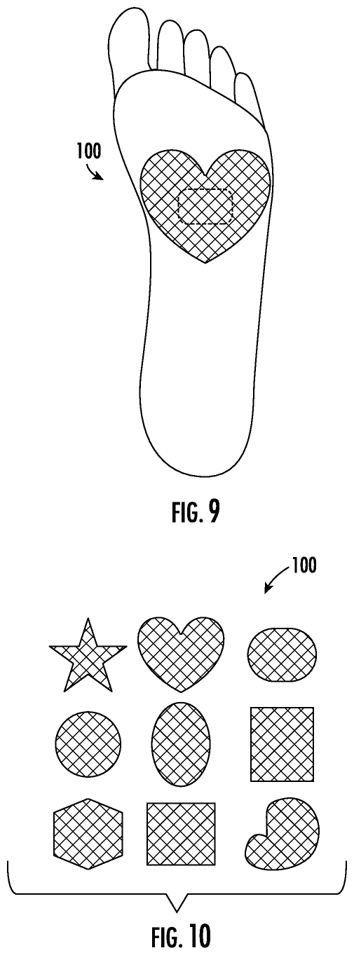

The foot patch 100 can have many different shapes and sizes and may include decorative elements and/or a decoratively-shaped outer perimeter 104. Additional examples of the foot patch 100 are provided in FIGS. 9 and 10.

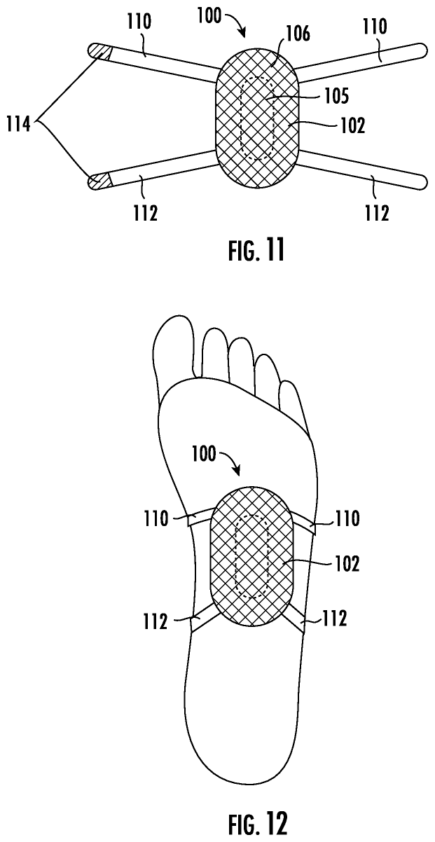

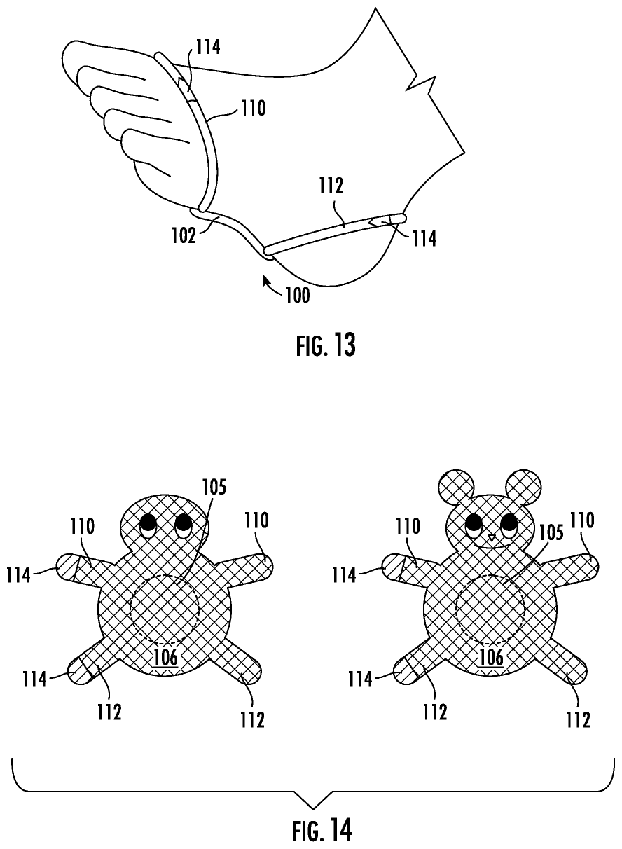

Yet another example of the foot patch 100 will now be described by referring to FIGS. 11-13. In this example, the foot patch 100 includes a pair of cooperating first straps 110 and a pair of cooperating second straps 112. The first straps 110 extend outwardly in opposing directions from the backing 102 and are adapted to wrap around the mid-foot region of the foot and attach together via an attachment mechanism 114. The second straps 112 extend outwardly in opposing directions from the backing 102 and are adapted to wrap around the heel region of the foot and attach together via another attachment mechanism 114. Together the first straps 110 and second straps 112 secure the foot patch 100 to the wearer's foot similar to the straps on sandals. This may be helpful for keeping the foot patch 100 in place when worn by an infant or young child or when worn while performing physical activity.

The attachment mechanism 114 may have many different forms so long as the attachment mechanism 114 used is capable of holding together the first straps 110 and second straps 112. Examples of suitable attachment mechanisms include adhesive, hook and loop fasteners, a knot, a buckle, and the like. The two attachment mechanisms 114 may be the same or different. One or both of the first straps 110 and second straps 112 may include an attachment mechanism 114. For example, adhesive may be on the top the second straps 112 and on the bottom of the first strap 110.

In the example of FIGS. 11-13, the skin contact surface 108 does not necessarily need to include the adhesive 107 because the first straps 110 and second straps 112 can secure the foot patch 100 to the foot. In some cases, however, it may be desirable to include the adhesive 107 on the skin contact surface 108 to provide an additional mechanism for holding the foot patch 100 in place on the foot.

As shown in FIG. 14, the foot patch 100 with first straps 110 and second straps 112 may also be designed in a decorative manner, such as by emulating the shape of an animal with the animal's arms and legs functioning as the first straps 110 and second straps 112.

Referring to FIG. 15, in another example, the first straps 110 and second straps 112 may be arranged to crisscross over the top of the foot such that the first straps 110, respectively, attach to the second straps 112.

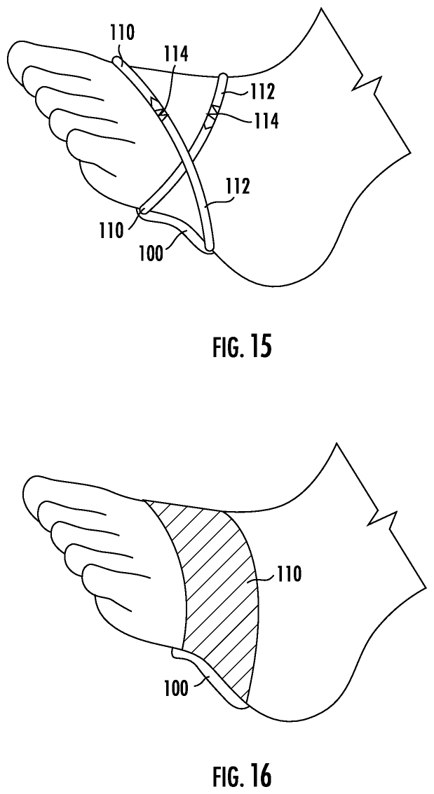

Referring to FIG. 16, in another example, the foot patch 100 may include a single first strap 110 that wraps around the foot.

The foot patch and related methods are not limited to the details and features described in connection with the example embodiments. There are numerous variations and modification of the compositions and methods that may be made without departing from the scope of what is claimed.

That which is claimed is:

1. A method comprising treating cough in a human by applying a therapeutic foot patch to a bottom of the human's foot, the therapeutic foot patch including:
a flexible backing having an adhesive skin contact side that is adhered to the bottom of the foot;
an active ingredient carrier section on the adhesive skin contact side, the active ingredient carrier section carrying a cough-reducing active ingredient that imparts a chemesthetic sensation to the foot.

2. The method of claim 1, wherein the active ingredient carrier section does not contact a heel of the foot or balls of the foot.

3. The method of claim 1, wherein the flexible backing does not contact a heel of the foot or balls of the foot.

4. The method of claim 1, wherein the human is a child.

5. The method of claim 1, wherein the cough-reducing active ingredient is within a liquid absorbent material on the active ingredient carrier section.

6. The method of claim 1, wherein the therapeutic foot patch is applied to a bottom of the human's big toe.

7. The method of claim 1, wherein the adhesive skin contact side includes adhesive along an outer perimeter of the therapeutic foot patch and the active ingredient carrier section is surrounded by the adhesive.

8. The method of claim 1, further comprising, immediately prior to applying the therapeutic foot patch to the bottom of the human's foot, placing the cough-reducing active ingredient on the active ingredient carrier section.

9. The method of claim 1, further comprising strapping the therapeutic foot patch to the human foot by wrapping a strap of the therapeutic foot patch around a midfoot region of the foot.

10. The method of claim 1, wherein the active ingredient carrier section is on a medial longitudinal arch of the foot when applied to the foot.

11. A method comprising treating cough in a human by applying a therapeutic foot patch to a bottom of the human's foot, the therapeutic foot patch including:
a flexible backing having a non-adhesive skin contact side that contacts the bottom of the foot;
an active ingredient carrier section on the non-adhesive skin contact side, the active ingredient carrier section carrying a cough-reducing active ingredient that imparts a chemesthetic sensation to the foot; and
at least one strap that wraps around the foot and attaches the therapeutic foot patch thereto.

12. The method of claim 11, wherein the active ingredient carrier section does not contact a heel of the foot or balls of the foot.

13. The method of claim 11, wherein the flexible backing does not contact a heel of the foot or balls of the foot.

14. The method of claim 11, wherein the human is a child.

15. The method of claim 11, wherein the cough-reducing active ingredient is within a liquid absorbent material on the active ingredient carrier section.

16. The method of claim 11, further comprising, immediately prior to applying the therapeutic foot patch to the bottom of the human's foot, placing the cough-reducing active ingredient on the active ingredient carrier section.

17. The method of claim 11, further comprising strapping the therapeutic foot patch to the human foot by wrapping a strap of the therapeutic foot patch around a midfoot region of the foot.

18. The method of claim 11, wherein the active ingredient carrier section is on a medial longitudinal arch of the foot when applied to the foot.

19. The method of claim 11, wherein the at least one strap includes a pair of straps that wrap around the foot and attach the therapeutic foot patch thereto.

20. The method of claim 11, wherein the therapeutic foot patch forms a shape of an animal with arms and legs and the arms and legs form the at least one strap.
